(19) United States
(12) Patent Application Publication
(10) Pub. No.: US 2002/0111457 A1
(43) Pub. Date: Aug. 15, 2002

(54) Reaction products of melamine and derivatives thereof

(76) Inventor: Wei Li, Mason, OH (US)

Correspondence Address: COGNIS CORPORATION, 2500 RENAISSANCE BLVD., SUITE 200, GULPH MILLS, PA, 19406

(21) Appl. No.: 10/015,523

(22) Filed: Dec. 13, 2001

Related U.S. Application Data
(60) Provisional application No. 60/256,375, filed on Dec. 18, 2000.
(60) Provisional application No. 60/314,781, filed on Aug. 24, 2001.

(51) Int. Cl. C08G 12/30; C07D 251/54

(52) U.S. Cl. 528/254; 544/196

(57) ABSTRACT

Reaction products of the reactants comprising

a) an intermediate reaction product of melamine and formaldehyde; and

b) at least one compound of formula I

RX(EO)ₙ(PO)ₘ(BO)ₚH (I)

wherein R is a substituted or unsubstituted, saturated or unsaturated, organic group having from 4 to 36 carbon atoms; X is —O—, —S—, or —NR¹— where R¹ is hydrogen or a C₁-C₈ alkyl group; n is a number of from 0 to 100; m is a number of from 0 to 50; and p is a number of from 0 to 50; provided that the sum of n, m, and p is at least 1; wherein the EO represents the residue of ethylene oxide, PO represents the residue of propylene oxide, and BO represents the residue of butylene oxide; and wherein the EO, PO, and BO groups when present can be in random and/or block distribution and can be in any order with respect to the X group; wherein the above reaction products are useful as low foaming surfactants and/or as defoaming agents.

### CROSS REFERENCE TO RELATED APPLICATIONS

This application claims the benefit of copending provisional application serial No. 60/256,375, filed on Dec. 18, 2000, and provisional application serial No. 60/314,781 filed on Aug. 24, 2001; the entire contents of each of which are incorporated herein by reference.

### FIELD OF THE INVENTION

This invention relates to compounds useful as low foaming surfactants and as defoaming agents in aqueous and nonaqueous liquid compositions.

### BACKGROUND OF THE INVENTION

Aqueous cleaning compositions exhibit a tendency toward foaming since they contain surface active agents such as soaps, and synthetic detergents. In many instances, such cleaning compositions produce excessive foam and the user must add substances known as anti-foaming agents or defoamers. Some defoamers such as silicones tend to interfere with the function of the cleaning compositions in that unwanted residues are left after the cleaners are wiped off, while others are environmentally unacceptable because they are not biodegradable.

Alkyl polyglycosides are a class of nonionic surfactants that exhibit significantly higher foaming profiles than other nonionic surfactants, such as alcohol ethoxylates. In fact, the foaming tendencies of alkyl polyglycosides more closely resemble those of anionic surfactants, such as alcohol sulfates, than the foaming tendencies of other nonionic surfactants. This higher foaming tendency makes the use of alkyl polyglycosides alone undesirable for many applications, e.g. cleaning-in-place for food processing plants, high pressure spray cleaning, bottle washing, floor cleaners, and automatic dishwashing, wherein high levels of foam interfere with the cleaning and rinsing operation and reduce the efficiency of the operation.

Low foam nonionics, such as EO/PO block copolymers, can be used to reduce the foaming properties of alkyl polyglycosides and anionic surfactants, but these materials have undesirable properties, e.g. low biodegradability, relatively high aquatic toxicity, and poor caustic compatibility.

Accordingly, there is a need for the development of defoamers that do not interfere with the cleaning ability of aqueous cleaning compositions and that are biodegradable, exhibit low aquatic toxicity, and good caustic compatibility.

There is also a need for defoamers for nonaqueous compositions.

In addition, there is a continuing need for low foaming surfactants for use in both aqueous and nonaqueous compositions.

### SUMMARY OF THE INVENTION

This invention relates to the reaction products of melamine, formaldehyde, and alkoxylated compounds which are useful as low foaming surfactants and as defoaming agents in aqueous and nonaqueous liquid compositions.

In particular, the reaction products of the invention are the reaction products of

a) an intermediate reaction product of melamine and formaldehyde, and

b) at least one compound of formula I

RX(EO)ₙ(PO)ₘ(BO)ₚH (I)

wherein R is a substituted or unsubstituted, saturated or unsaturated, organic group having from 4 to 36 carbon atoms; X is —O—, —S—, or —NR¹— where R¹ is hydrogen or a C₁-C₈ alkyl group; n is a number of from 0 to 100, e.g. from 1 to 100; m is a number of from 0 to 50 e.g., from 1 to 50; and p is a number of from 0 to 50 e.g., from 1 to 50; provided that the sum of n, m, and p is at least 1; wherein EO represents the residue of ethylene oxide, PO represents the residue of propylene oxide, and BO represents the residue of butylene oxide; and wherein the EO, PO, and BO groups when present can be in random and/or block distribution and can be in any order with respect to the X group.

### DESCRIPTION OF THE PREFERRED EMBODIMENTS

Other than in the operating examples, or where otherwise indicated, all numbers expressing quantities of ingredients or reaction conditions used herein are to be understood as modified in all instances by the term “about”. Also, throughout this description, unless expressly stated to the contrary; percent “parts” of, and ratio values are by weight; the description of a group or class of materials as suitable or preferred for a given purpose in connection with the invention implies that the mixtures of any two or more of the members of the group or class are equally suitable or preferred; description of constituents in chemical terms refers to the constituents at the time of addition to any combination specified in the description or of generation in situ by chemical reactions specified in the description, and does not necessarily preclude other chemical interactions among the constituents of a mixture once mixed.

The R group in formula I can be any substituted or unsubstituted, saturated or unsaturated organic moiety having from 4 to 36 carbon atoms. Thus the R groups can be linear or branched alkyl groups, linear or branched alkenyl or alkynyl groups, saturated carbocyclic moieties, unsaturated carbocyclic moieties having one or more multiple bonds, saturated heterocyclic moieties, unsaturated heterocyclic moieties having one or more multiple bonds, substituted linear or branched alkyl groups, substituted linear or branched alkenyl or alkynyl groups, substituted saturated carbocyclic moieties, substituted unsaturated carbocyclic moieties having one or more multiple bonds, substituted saturated heterocyclic moieties, substituted unsaturated heterocyclic moieties having one or more multiple bonds, and aryl or arenyl moieties. Arenyl groups are alkyl-substituted aromatic radicals having a free valence at an alkyl carbon atom such as a benzylic group. Aryl groups include those having from 6 to 36 carbon atoms, e.g., phenyl, naphthyl, etc.

Examples of R groups include but are not limited to substituted or unsubstituted alkyl groups having from 4 to 36 carbon atoms, preferably from 4 to 22 carbon atoms, more preferably from 4 to 12 carbon atoms, and most preferably from 8 to 10 carbon atoms; alkenyl and alkynyl groups having from 4 to 36 carbon atoms, preferably from 4 to 22 carbon atoms, aryl groups having from 6 to 26 carbon atoms, and arenyl groups having from 7 to 36 carbon atoms. When the R groups are substituted groups, the groups can contain single or multiple substitutions such as a halogen substitution, for example Cl, Fl, I, and Br; a sulfur functionality such as a mercaptan or thio group; a nitrogen functionality such as an amine or amide functionality; a silicon functionality; a C₁-C₄ alkoxy group; or any combination thereof.

In the formula I compounds, compounds wherein the sum of n, m, and p is at least 2, especially at least 3, are preferred.

The degree of ethoxylation is preferably from 2 to about 50, more preferably from 3 to about 50, with the most preferred being from 4 to 50, while the degree of propoxylation and butoxylation can vary from 0 to about 50, e.g. from 0 to about 10. The degree of propoxylation and/or butoxylation will be determined by the desired degree of water solubility and miscibility. The water solubility and miscibility will ultimately be determined by such factors as the number of carbon atoms in R, the relative amounts of EO, PO, and BO, and the effect of PO and BO on the biodegradability of the compound. The water solubility and miscibility of a compound according to the invention and the interrelationships between the number of carbon atoms in R group, the relative amounts of EO, PO, and BO and the biodegradability of the final product will be readily determinable by one of ordinary skill in the art.

When the X group is —NR¹—, the compounds of formula I are alkoxylated amines. When the compounds of formula I are alkoxylated amines, n is a number from 0 to 50, preferably from 1 to 50, m is a number from 0 to 50 and p is a number from 0 to 50, preferably from 1 to 50, provided that the sum of n, m, and p is at least 1. Examples of the alkoxylated amines useful for the purposes of the invention include but are not limited to, alkoxylated dibutyl amine, alkoxylated dicyclohexyl amine, alkoxylated diethylethanolamine, and alkoxylated dioctylamine.

Preferred compounds of formula I have the following formula II

RO(EO)ₙ(PO)ₘ(BO)ₚH (II)

in which R, n, m, and p have the meanings given above.

In the compounds of formulae I and II, as stated above for the compounds of formula I, it is understood that EO stands for the residue of ethylene oxide, PO stands for the residue of propylene oxide, and BO stands for the residue of butylene oxide. Also, in the compounds of formulae I and II, the EO, PO, and BO groups, when present, can be in any order with respect to the RX or RO group, and can be in blocks and/or random distribution, although the alkoxide groups present are preferably present in the order shown in formulae I and II.

The reaction between reactants a) and b) can be carried out by reacting the components at a temperature in the range of from 90 to 150° C., preferably from 90 to 110° C. The reaction can be carried out using a catalyst, e.g., borax, preferably in an alkaline solution. The reaction is preferably carried out in an inert atmosphere such as a nitrogen atmosphere.

The intermediate reaction product a), i.e. the reaction product of melamine and formaldehyde, can be prepared by reacting melamine with formaldehyde or paraformaldehyde in a basic aqueous solution at a temperature in the range of from 50 to 100° C., preferably from 75 to 85° C. The equivalent ratio of formaldehyde or paraformaldehyde to the amino groups in melamine can range from 6:1 or more to 2:1, preferably from 4:1 to 2:1. The reaction proceeds as follows to give an intermediate product of formula III

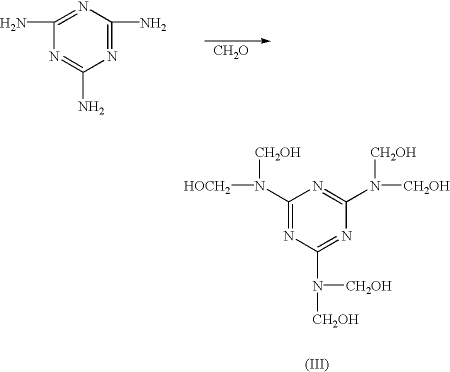

Intermediate product III is then reacted with a compound of formula I in the presence of an acid to give a product of formula IV below:

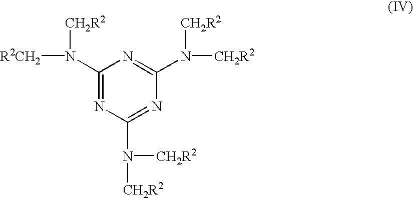

where each R² is independently —OH or RX(EO)ₙ(PO)ₘ(BO)ₚ—, where R, X, n, m, and p have the meanings given above; provided that at least one R² group is an RX(EO)ₙ(PO)ₘ(BO)ₚ— group. It is preferred that at least three R² groups and more preferably all six R² groups are RX(EO)ₙ(PO)ₘ(BO)ₚ— groups.

In the reaction between components a) and b), the equivalent ratio of component b) to the hydroxy groups of component a) can range from 3:1 or more to 1:6, preferably from 3:1 to 1:1. A product of formula IV above in which all R² groups are RX(EO)ₙ(PO)ₘ(BO)ₚ− is obtained when the above ratio is from at least 3:1 to 1:1. When the ratio is less than 1:1, i.e. when fewer equivalents of the compound of formula I than are required to react with all of the hydroxy groups of the component a) intermediate reaction products are present, then one or more of the R² groups remain unreacted, i.e. remain as —OH groups. When the above ratio is less than 1:1, mixtures of compounds of formula IV will usually result.

The reaction products of the invention can be used as low foaming surfactants in both aqueous and nonaqueous compositions in surfactant-effective amounts, usually from 0.1 to 10% by weight, preferably from 1 to 5% by weight, based on the weight of the composition.

These reaction products can also be used in the above quantities as defoaming agents for aqueous and nonaqueous compositions, and are particularly useful in minimizing or eliminating foaming in aqueous compositions containing high foaming surfactants, such as alkyl polyglycosides and anionic surfactants such as alcohol sulfates.

These reaction products can be used in aqueous cleaning compositions, in emulsion polymer latex compositions such as latex paints, in inks, in adhesives, in metal working compositions, and in other aqueous and nonaqueous compositions in which surfactants and/or defoaming agents are advantageously present.

The reaction products of the invention are biodegradable, contain no organic solvents, and do not adversely affect the detergency of other surfactants that may be present in the compositions in which they are used since they are themselves surfactants.

The invention will be illustrated by the following example.

### EXAMPLE

Synthesis of Hexamethylol melamine and POE (6) Tridecyl Alcohol Adduct.

A 37% aq. solution of formaldehyde (28.40 g, 0.35 mole) was charged into a 250 ml three neck flask, which was equipped with a distillation head apparatus, a nitrogen inlet, and a thermometer. 50 wt. % NaOH aqueous solution was added dropwise with stirring until a pH 10.5 was obtained. Melamine (6.3 g, 0.05 mole) was added. The reaction mixture was heated up to 80° C. for 30 min., then 50° C. for 3 hrs. Without purification, POE (6) Tridecyl alcohol (102.3, 0.22 mole) and borax (0.3 g, as catalyst) were added. The mixture was heated up to 100° C. until no more water was obtained (about 3 hrs.).

What is claimed is:

1. A reaction product of the reaction between reactants comprising
a) a reaction product of melamine and formaldehyde; and
b) at least one compound of formula I
RX(EO)ₙ(PO)ₘ(BO)ₚH (I)
wherein R is a substituted or unsubstituted, saturated or unsaturated, organic group having from 4 to 36 carbon atoms; X is —O—, —S—, or —NR¹— where R¹ is hydrogen or a C₁-C₈ alkyl group; n is a number of from 0 to 100; m is a number of from 0 to 50; and p is a number of from 0 to 50; provided that the sum of n, m, and p is at least 1; wherein EO represents the residue of ethylene oxide, PO represents the residue of propylene oxide, and BO represents the residue of butylene oxide; and wherein the EO, PO, and BO groups when present can be in random and/or block distribution and can be in any order with respect to the X group.

2. The reaction product of claim 1 wherein in component b) the sum of n, m, and p is at least 2.

3. The reaction product of claim 2 wherein the sum of n, m, and p is at least 3.

4. The reaction product of claim 1 wherein in component b), R is an alkyl group containing from 4 to 12 carbon atoms.

5. The reaction product of claim 4 wherein the alkyl group contains from 8 to 10 carbon atoms.

6. The reaction product of claim 1 wherein in component b), X is —O—.

7. The reaction product of claim 1 wherein in component b), X is —NR¹—.

8. The reaction product of claim 1 wherein in component b), n is a number of from 2 to about 50.

9. The reaction product of claim 1 wherein component b) has the formula
RO(EO)ₙ(PO)ₘ(BO)ₚH (II)
where n=4 to 50, and R, m, and p have the meanings given in claim 1.

10. The reaction product of claim 9 wherein R is an alkyl group containing from 4 to 12 carbon atoms.

11. The reaction product of claim 9 wherein the alkoxide groups present are present in the order shown in formula II.

12. The reaction product of claim 1 wherein in component b) the alkoxide groups present are present in the order shown in formula I.

13. The reaction product of claim 1 wherein the x group in formula I is a number of from 3 to 6.

14. A compound of formula IV below: 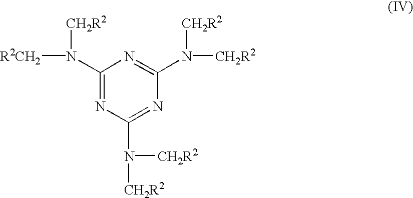
where each R² is independently —OH or RX(EO)ₙ(PO)ₘ(BO)ₚ—, wherein R is a substituted or unsubstituted, saturated or unsaturated, organic group having from 4 to 36 carbon atoms; X is —O—, —S—, or —NR¹— where R¹ is hydrogen or a C₁-C₈ alkyl group; n is a number of from 0 to 100; m is a number of from 0 to 50; and p is a number of from 0 to 50; provided that the sum of n, m, and p is at least 1; wherein EO represents the residue of ethylene oxide, PO represents the residue of propylene oxide, and BO represents the residue of butylene oxide; and wherein the EO, PO, and BO groups when present can be in random and/or block distribution and can be in any order with respect to the X group.

15. The compound of claim 14 wherein from 3 to 6 R² groups are RX(EO)ₙ(PO)ₘ(BO)ₚ— groups.

16. The compound of claim 14 wherein the sum of n, m, and p in each RX(EO)ₙ(PO)ₘ(BO)ₚ— group is at least 2.

17. The compound of claim 14 wherein each R group present therein is an alkyl group containing from 4 to 12 carbon atoms.

18. The compound of claim 17 wherein the R groups each contain from 8 to 10 carbon atoms.

19. The compound of claim 14 wherein X is in —O— each RX(EO)ₙ(PO)ₘ(BO)ₚ— group.

20. The compound of claim 14 wherein in each RX(EO)ₙ(PO)ₘ(BO)ₚ— group present therein, n is a number of from 2 to about 50.

21. The compound of claim 14 wherein the alkoxide groups present therein are present in the order shown in formula IV.

22. The compound of formula III below:

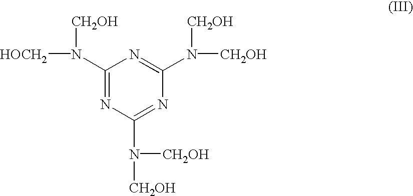

23. In an aqueous composition, the improvement wherein a surfactant-effective or defoaming-effective quantity of the reaction product of claim 14 is present therein.

24. The composition of claim 23 wherein the surfactant-effective or defoaming-effective quantity is from about 0.1 to about 10% by weight.

25. The composition of claim 23 wherein the composition is a latex paint composition.

26. In an aqueous composition containing an alkyl polyglycoside and/or an alcohol sulfate the improvement wherein a defoaming-effective quantity of the reaction product of claim 14 is present therein.

27. In a nonaqueous composition, the improvement wherein a surfactant-effective or defoaming effective quantity of the reaction product of claim 14 is present therein.

28. The nonaqueous liquid composition of claim 27 wherein the composition is an ink, an adhesive, or a metal working composition.

29. The nonaqueous liquid composition of claim 27 wherein the surfactant-effective quantity is from about 0.1 to about 10% by weight.
